(19) United States
(12) Patent Application Publication
(10) Pub. No.: US 2026/0004871 A1
(43) Pub. Date: Jan. 1, 2026

(54) CELL TREE RINGS: METHOD AND CELL LINEAGE TREE BASED AGING TIMER FOR CALCULATING BIOLOGICAL AGE OF BIOLOGICAL SAMPLE TECHNICAL FIELD

(71) Applicant: AgeCurve Limited, Cambridge (GB)

(72) Inventors: Attila Csordas, Cambridge (GB); Botond Sipos, Cambridge (GB); Damien Hicks, Cambridge (GB)

(21) Appl. No.: 19/110,666

(22) PCT Filed: Sep. 13, 2023
(86) PCT No.: PCT/IB2023/059080

§ 371 Date: Mar. 11, 2025

(30) Foreign Application Priority Data
Sep. 13, 2022 (GB) 2213358.1

(51) Int. Cl. G16B 10/00; C12N 15/10; G16B 20/20; G16B 40/00

(52) U.S. Cl. CPC G16B 10/00; C12N 15/1082; G16B 20/20; G16B 40/00

(57) ABSTRACT

In general terms the present invention proposes a method for calculating a biological age of a biological sample. The method comprises collecting the biological sample from a subject; preparing a single-cell RNA (scRNA) sequencing library from the biological sample, wherein preparing the scRNA sequencing library comprises a modified polymerization time and a modified purification process comprising two or more purification steps: identifying at least one type of genetic difference in the biological sample from the single cell RNA (scRNA) sequencing library; and gencrating a cell lineage tree that reflects a branching order of cells of the biological sample, wherein the branching order, with branch lengths included, of cells provides a data set for validating the biological age of the biological sample. The present invention also proposes a cell lineage tree based aging timer for calculating a biological age of a biological sample using the aforementioned method.

This invention relates to a biological age estimation method. In particular, though not exclusively, this invention relates to a method for calculating a biological age of a biological sample. Moreover, this invention also relates to a cell lineage tree-based aging timer for calculating a biological age of a biological sample.

### BACKGROUND

Aging is a complex process marked by a steady deterioration in physical, mental, and reproductive capacities, which leads to a loss of function, increased susceptibility to disease, and, eventually, death. Therefore, a reliable calculation of biological age is required for the estimation of healthy longevity interventions targeting the root causes of the aging process that is useful in other clinical interventions, for instance, cell therapy and the like.

Several techniques have been used to calculate a biological age-rather than the chronological age—of a subject, e.g. a person. Typically, an aging clock (or aging timer) reflecting the life history of a subject is used for the calculation of the biological age. However, the existing techniques fail to provide an efficient and a reliable molecular/cellular aging clock to reflect directly the life history of the person. A general problem of finding a reliable molecular/cellular aging clock critically relies on the biological sample (such as blood, plasma, serum, saliva DNA, and so on) that the aging clock operates on. Moreover, the reliable molecular/cellular aging clock also relies on whether said biological sample can be scaled up to include significant portions of the human body to reflect a big enough portion, thereby offering mechanistic explanations working at the tissue level.

Conventional aging clocks are based on epigenetic aging patterns measuring CpG methylation islands on bulk DNA. Such aging clocks do not offer good mechanistic explanations and can only provide an epigenetic aging trajectory probabilistically that does not provide information on the same biological object that directly reflects the subject's temporal history. There also exists a technical problem of how to calculate the biological age of the subject with improved efficiency, reproducibility, reliability, scalability and cost effectiveness.

There remains a need for improved methods and novel systems for calculating the biological age of a biological sample.

### SUMMARY OF THE INVENTION

A first aspect of the invention provides a method for calculating a biological age of a biological sample, the method comprising:
collecting the biological sample from a subject;
preparing a single-cell RNA (SCRNA) sequencing library from the biological sample, wherein preparing the scRNA sequencing library comprises a modified polymerization time and a modified purification process comprising two or more purification steps;
identifying at least one genetic difference in the biological sample from the single-cell RNA (scRNA) sequencing library
establishing filters to maximise true positive somatic mutation calls in the single-cell RNA (SCRNA) sequencing library;
generating a cell lineage tree that reflects a branching order of cells of the biological sample, wherein the branching order of cells provides a data set for calculating the biological age of the biological sample.

Herein, the disclosed method enables calculating the biological age from the biological samples obtained from the subject(s) by preparing the single-cell RNA (SCRNA) sequencing library from the biological samples. The single-cell RNA (scRNA) sequencing library are, for example, 10× genomics scRNA sequencing libraries. The method provides identification of at least one type of genetic difference, such as somatic single nucleotide variants (SNVs), in the biological sample to generate the cell lineage tree to provide an efficient and reliable calculation of biological age.

Throughout the present disclosure, the terms “tree feature”, “predictor” and “predictor variable” are used interchangeably. These terms are discussed in further detail below.

Throughout the present disclosure, the term “biological sample” as used herein refers to a test material from the subject that is used for assaying one or more properties. Herein, the subject may be a human. In an embodiment, the biological sample is selected from any of: a blood sample, a saliva sample, a cell, a tissue, any other analyte. Herein, the biological sample serves as a source of RNA or DNA of the subject to test for any potential or existing disease condition in the subject. Optionally, the biological sample is a peripheral or capillary blood comprising thousands to millions of cells. It will be appreciated that the biological sample is collected at a dedicated site, such as a clinic or a laboratory, and is processed under sterile conditions. In this regard, the peripheral blood may be collected from the median cubital vein of the subject at a clinic with properly trained personnel. Moreover, the term “biological age” of the biological sample as used herein refers to a rate at which people grow old and how old their bodies seem to be. Particularly, the biological age computed here is a Cell Tree Age, and the terms biological age, Cell Tree Age or biological Cell Tree Age are used interchangeably therein. It will be appreciated that the biological age may depend on a number of factors, such as the change in chromosomes over time, DNA methylation (resulting from exposure to sunlight, exhaust gases, alcohol, chemicals, and so on), damage to various cells and tissues, diseases, lifestyle, nutrition, chronological age, and so forth. Notably, the chronological age is the total amount of time (in days, months or years) of existence of the subject irrespective of the aforementioned factors that influence the biological age. The method of the invention comprises preparing a single-cell RNA (ScRNA) sequencing library from the biological sample. The term “single-cell RNA (scRNA) sequencing library” refers to sequencing library in which transcriptomes, i.e. a total RNA content (including protein-coding RNAs (such as mRNA) and regulatory or non-coding RNS (such as miRNA, tRNA, and the like), in a single cell of the biological sample are mapped to individual cells, using a scRNA sequencing (scRNA seq) tool. Notably, scRNA sequencing library preparation is usually done to analyse gene expression to identify which genes are turned on or off in a given biological sample. Optionally, the single cell RNA (SCRNA) sequencing library is a 10× Genomics single-cell RNA (SCRNA) sequencing library. Beneficially, 10× Genomics single-cell RNA (SCRNA) sequencing library provide increased sample throughput and increased number of cells assayed in a single experiment

In an embodiment, the scRNA sequencing library comprises at least one of: a gene expression library, a cell surface protein library, any other library, for generating a high-resolution cell lineage tree downstream. Typically, an expression library may be a collection of DNA, RNA or protein products. Specifically, the gene expression library is a library of DNA fragments created with expression vectors for expressing genes in the library. Typically, the cell surface protein library is prepared by labeling cell surface proteins using a specific protein binding molecule, such as an antibody conjugated to a Feature Barcode Oligonucleotide.

Moreover, preparing the scRNA sequencing library comprises a modified polymerization time and a modified purification process comprising two or more purification steps. In this regard, the polymerization time for cDNA amplification is extended from conventional polymerization time. In an embodiment, the modified polymerization time is in a range of 1-2 minutes. Optionally, in an implementation the polymerization time for cDNA amplification is extended from 1 minute to 1.5 minute. Moreover, the modified purification process comprises three purification steps as compared to conventional two purification steps. In this regard, two different Solid Phase Reversible Immobilization (SPRI) steps and transfer volumes have been applied after the cDNA purification step in after-fragmentation step. Notably, SPRI is advantageous for low concentration DNA clean-up. Beneficially, the modified polymerization time and the modified purification process generates a better quality and quantity of the scRNA sequencing library, preferably, a 10× Genomics scRNA sequencing library. Moreover, during the preparation of the scRNA sequencing library, the minimum number of cells barcoded per sample is approximately 1000 cells and the increased number of reads per sample and cell is more than 60 million reads per samples (e.g between 60 and 180 million reads per sample) and more than 60 thousand reads per cell. Moreover, the fragment size of the sequenced cDNA also increased resulting from the aforementioned modifications. It will be appreciated that the 10× Genomics scRNA sequencing library is generated in a format, such as BAM or SAM, that stores sequence reads mapped to reference sequences. Notably, BAM is a binary file that may not be readable by most bioinformatics pipelines. Therefore, such a preliminary BAM file needs to be converted into a suitable format, such as a FASTQ format file, that contains sequence data before mapping it to the reference sequences. Moreover, the method comprises converting the obtained BAM files into a corresponding FASTQ format file using a ‘bamtofastq’ pipeline (or tool). Ideally, the FASTQ format file is a predecessor of a BAM file which is a result of merging many individual FASTQ format files. Notably, the FASTQ format file is a text-based file for storing sequence data along with their corresponding quality scores. Typically, the FASTQ format files have the same set of sequences that were input via the BAM file. Moreover, the FASTQ format files contain a sequence of quality scores associated with each nucleotide in the sequences therein. Herein, the term “quality score” refers to a weight, value or rank associated with the sequences in the FASTQ format files. Moreover, the method comprises identifying at least one type of genetic difference in the biological sample from the single-cell RNA (SCRNA) sequencing library. Herein the term “identifying” may be understood as ‘calling’ genetic differences from the scRNA sequencing library using a range of methods or bioinformatics pipelines or tools.

The term “genetic difference” as used herein refers to a difference in the genetic information (namely, genome) in and among populations. The genome comprises coding regions (or DNA) that code for proteins and non-coding regions. Notably, the genome includes the nuclear DNA, mitochondrial DNA and chloroplast DNA of an individual organism. Optionally, genetic differences may result from mutations, sexual reproduction and/or genetic drift. Moreover, genetic differences may be a single nucleotide polymorphism (SNP) or brief insertions or deletions (indels) in the genetic sequence. Notably, the SNPs and indels constitute more than 99.9% of the typical genetic differences from a reference human genome. Furthermore, such genetic differences may be associated with at least one disease or a phenotype of an individual.

It will be appreciated that at least one genetic difference may be a germline variation or a somatic variation. Herein the germline variation is hereditary and may be present in egg or sperm cells, and the somatic variation is non-hereditary and may be present in specific cells and may be acquired at some point during an individual's lifetime randomly, during cell divisions or as a result of environmental factors or lifestyle of the individual.

In an embodiment, at least one genetic difference is selected from somatic single nucleotide variants (SNVs) from a single-cell RNA (scRNA) sequencing library. Typically, SNVs are forms of SNPs that cause germline or somatic substitutions of a single nucleotide at a specific position in the genome (in coding sequences of genes or non-coding regions of genes) of an individual. Therefore, SNVs may be associated with particular biological traits and disease susceptibilities, such as cancers, age-related macular degeneration, non alcoholic fatty liver disease, Alzheimer's disease, and so on. It will be appreciated that the SNVs may not necessarily change the amino acid as a result of degeneracy of the genetic code), but may still affect the gene splicing, transcription factor (TF) binding activity, mRNA degradation, sequence of non-coding RNA, and the like.

Furthermore, the method comprises generating the cell lineage tree that reflects the branching order of cells of the biological sample, wherein the branching order of cells provides the data set for validating the biological age of the biological sample. The term “cell lineage tree”, and the abridged version “cell tree”, as used herein refers to a common evolutionary or developmental history of one or more cells or tissue from its originator (or progenitor) cells, i.e. a sperm, an egg, or an embryo. Typically, cell division and cell relocation forms the basis of study of an individual's cellular ancestry. The term “branching order of cells” as used herein refers to a development of a cell from an originator cell and finishing off as an existing, intact cell at the time of sampling the tissue. Cell lineage trees can be inferred with phylogenetic algorithms that provide approximations of the exact branching order of cells. Typically, the cell lineage tree can be inferred from single cell genotypes, i.e. the at least one type of genetic difference identified or called from the single-cell RNA (scRNA) sequencing library. The method to generate cell lineage trees from SCRNA seq libraries employs at least one of: a Tizkit pipeline and a Tiznit pipeline. Optionally, a bigger set of diverse and comprehensive cell lineage tree features may be used for picking up age related signals for reflecting different modalities of the process. These features either combine topology and branch length information together by default, or are only capturing them separately. It will be appreciated that the method works better with more cell samples and the best age prediction is achieved with around 700 cells used or more.

In an embodiment, the data set for calculating the biological age comprises at least one of: a population history, a temporal history. The “population history” as used herein refers to an average population of a specified group, such as a control group or a diseased or patient group. Notably, the population history as a data set comprises a set of individuals who share one or more characteristics under an observation study. The term “temporal history” as used herein refers to a data that represents a specific characteristic in time. Notably, the temporal history provides one or more characteristics under an observation study.

In an embodiment, the method employs at least one algorithm selected from: a SCRNA SNV mapping tool, a linear regression, a penalised multiple regression, a Laplacian transform, a Wavelet-based transform, a Fourier based transform, a distance matrix-based algorithm. Notably, at least one algorithm enables validation and improvement of the biological age estimation by the cell lineage trees. Notably, the penalized multiple regression algorithm employs regressing multiple tree features of a healthy cohort in a diverse age range to yield a calibration curve (line), based on which estimating the chronological and biological age of the biological sample is obtained. A key development is the use of a comprehensive list of tree features to capture all the properties that could turn out to be useful when estimating the biological Cell Tree Ages of the samples and capturing the relevant, but different properties of the tree. For this task not only features already described in the literature and used in different context than biological age estimation and in general different context than cell lineage trees but novel features have been developed. This way an explorative, unbiased, but also targeted set of tree features have provided elements of the multiple regression model. There are features from at least 6 different groups: i., Laplacian transform based features, ii., Clonality focused features that can be ii/a. Fourier transform based Features or ii/b., Entropy-based features iii., Branch Length focused features iv., Traditional Phylogenetics based features v., Distance matrix based features vi., Graph based features.

In an embodiment, each of the linear regression, the multiple regression and the Laplacian transform comprises an independent predictor variable and a dependent response variable. Herein, the independent predictor variable is a feature capturing the topology of the cell lineage tree and the dependent response variable is the chronological age of the biological sample. In an embodiment, validating the biological age of the biological sample is based on a calibration curve obtained using the data set. The core concept of building a new molecular ageing clock is to find new features based on high throughput measurements and regress chronological age against it. Due to the complexity and multimodality of the ageing process better predictors can be built using multiple regression techniques taking into account several different predictors. The predictor variables can be used to predict the chronological age of the sample based on a calibration curve. If the calibration of the curve is attained from a healthy cohort of people across a diverse age range, then the age estimation can be used to predict the biological age of the biological sample in question and to show the disconnect between the chronological age and the biological age of said biological sample. This estimation then can be used as an indication to look for interventions in case the disconnect shows accelerated aging status, i.e. the biological age estimation is higher than the chronological age. In an embodiment, the method further comprises identifying a root of the cell lineage tree. Notably, the at least one algorithm, specifically the Tiznit pipeline, by default generates an unrooted cell lineage tree that specifies the branching topology fully and computes all the branch lengths (the number of substitutions per variable site) but it does not assume a particular root of the tree. However, one or more additional algorithms may be used for rooting the cell lineage tree.

A second aspect of the invention provides a cell lineage tree based aging timer for calculating a biological age of a biological sample using the method according to the first aspect of the invention, the cell lineage tree based aging timer configured to identify at least one type of genetic difference in the biological sample based on a data set obtained from a branching order of cells of the biological sample. The disclosed cell lineage tree based aging timer operates on cell lineage trees reflecting mitotic branching order of a biological sample, preferably cells. The cell lineage tree based aging timer enables reconstruction of part of the temporal life history of the subject. Moreover, the population history of cells and the underlying somatic evolution of genetic differences, namely, the single nucleotide variants (SNVs) may be used to suggest the healthy longevity interventions that may help to slow down or mitigate the damages caused by the aging process. Moreover, the cell lineage tree based aging timer may offer mechanistic explanations of physiological events and may be theoretically or methodologically scaled up to cover much bigger parts of the subject's body by sampling a large amount of biological sample, such as a large number of cells, for example from different tissues from the subject.

In an embodiment, at least one genetic difference is selected from somatic single nucleotide variants (SNV) from a single-cell RNA (SCRNA) sequencing library prepared using the aforementioned method.

In an embodiment, the cell lineage tree based aging timer employs at least one algorithm selected from: a scRNA SNV mapping tool, a linear regression, a multiple regression, a Laplacian transform, Wavelet/-based transform, a Fourier-based transform, a distance matrix-based algorithm. In an embodiment, each of the linear regression and the multiple regression comprises an independent predictor variable and a dependent response variable.

In an embodiment, the cell lineage tree based aging timer further comprises employing the at least one algorithm to process the data set and represent the calculated biological age of the biological sample as a graphical representation. In an embodiment, the data set for calculating the biological age comprises at least one of: a population history, a temporal history.

In an embodiment, validating the biological age of the biological sample is based on a calibration curve obtained using the data set.

In an embodiment, the scRNA sequencing library comprises at least one of: a gene expression library, a cell surface protein library, any other library for generating a high-resolution cell lineage tree, wherein the high-resolution cell lineage tree is used for producing the cell lineage tree based aging timer.

In an embodiment, the biological sample is selected from any of: a blood sample, a saliva sample, a DNA sample.

A third aspect of the invention provides a computer program product comprising a non-transitory computer-readable storage medium having computer-readable instructions stored thereon, the computer-readable instructions being executable by a computing device comprising a processor to execute the method according to the first aspect of the invention.

Optionally, the computer program product is implemented as an algorithm, embedded in a software stored in the non-transitory computer-readable storage medium. The non-transitory computer-readable storage medium may include, but is not limited to, an electronic storage device, a magnetic storage device, an optical storage device, an electromagnetic storage device, a semiconductor storage device, or any suitable combination of the foregoing. Examples of implementation of computer-readable storage medium, but are not limited to, Electrically Erasable Programmable Read-Only Memory (EEPROM), Random Access Memory (RAM), Read Only Memory (ROM), Hard Disk Drive (HDD), Flash memory, a Secure Digital (SD) card, Solid-State Drive (SSD), a computer readable storage medium, and/or CPU cache memory.

Throughout the description and claims of this specification, the words “comprise” and “contain” and variations of the words, for example “comprising” and “comprises”, mean “including but not limited to”, and do not exclude other components, integers or steps. Moreover, the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application, it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 is an illustration of the results of the ReproSignal Pipeline used to select those Cell Tree features that show much higher biological variation between individual samples compared to technical variation within the same individual sample across different replicates, in accordance with an embodiment of the present disclosure; and

FIG. 2 is an illustration depicting graphical representations of an estimation of biological Cell Tree Age of various subjects, in accordance with an embodiment of the present disclosure.

### DETAILED DESCRIPTION

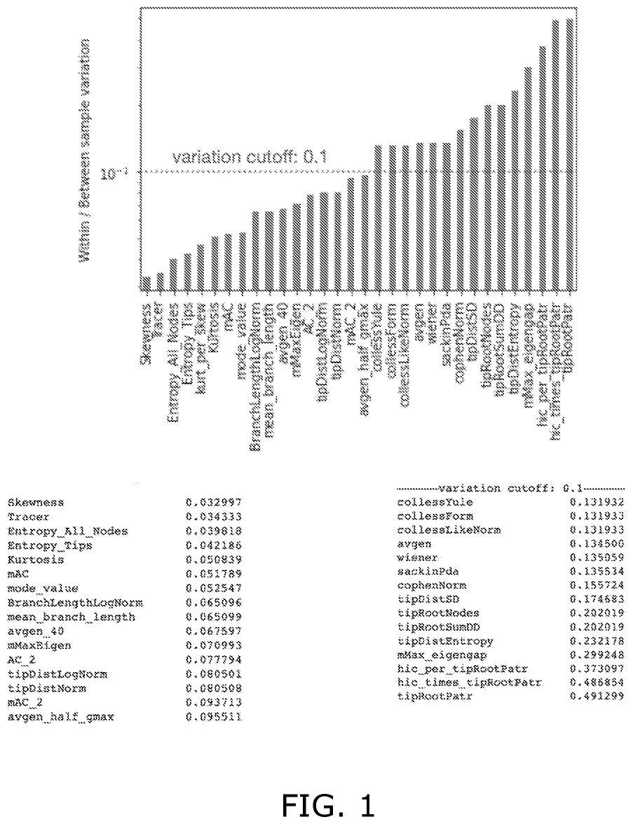

As shown in FIG. 1, the ReproSignal Pipeline is used to select those tree features that show at least 10 times higher biological variation between individual samples compared to technical variation within the same individual sample across different replicates. FIG. 1 shows a bar chart sorted according to descending within/between sample variation ratios on the Y-axis. The X axis shows the different tree features. The cutoff dotted red line is at 0.1. Under the bar chart are shown the same list of tree features and the actual numerical within/between sample variation ratios separated by the variation cutoff.

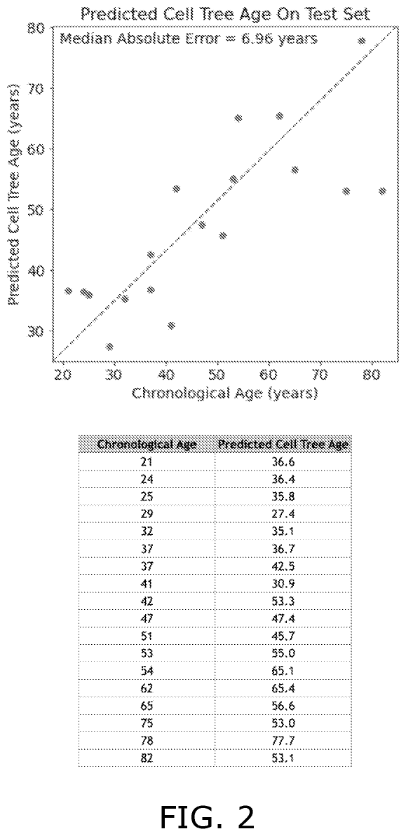

Referring now to FIG. 2. FIG. 2 shows a scatter plot of the Cell Tree Age test prediction on 18 human blood samples. The x axis is Chronological Age in years, and the y axis shows the numerical values of the predicted Cell Tree Ages in years. The red diagonal dashed line is for display purposes only. The error term indicated is the Median Absolute Error, and the numerical value of the error is 6.96 years. Under the scatter plot there is a table showing the Chronological Ages and the actual predicted Cell Tree Ages of the test individuals expressed in years.

### Experimental Data

Biological sample collection and isolation of cells therefrom: Viable peripheral blood mononuclear cells (PBMCs) or cells (as used hereafter) were isolated from the collected biological sample. In this regard, 4 ml of peripheral blood was diluted with 4 ml of 2% Fetal bovine serum (FBS) or Phosphate buffered saline (PBS), and the rest of the peripheral blood was frozen for DNA isolation therefrom. Subsequently, 8 ml of diluted peripheral blood was carefully layered on top of 4 ml of a density gradient (such as Lymphoprep™) and centrifuged at 300 g for 30 min. The cells were carefully harvested from the interface with a plastic pasteur pipette. Then, another 6 ml of 2% FBS/PBS is added to the cells, and then centrifuged at 300 g for 8 min with discarding of the supernatant and resuspending the cells in 1 ml of lysis solution. After one minute incubation on ice, 4 ml of 2% FBS/PBS was added to the cells and centrifuged at 300 g for 5 min with discarding of the supernatant and resuspending the cells in 1 ml of 2% FBS/PBS. Subsequently, the vitality and concentration of cells was determined through Acridine Orange and Propidium Iodide assay at LUNA Automated Cell Counter. Table 1 below provides data corresponding to vitality and concentration of cells post isolation from the biological sample.

TABLE 1
Vitality and concentration of cells post isolation

| | | | μl into | Cell |
|---|---|---|---|---|
| | Concentration | Viability | CellPlex | Multiplexing |
| Sample | [b/ml] | [%] | staining | Oligo |
| HLC 1 | _{5.08×10}6 | 99.1 | 400 | 304 |
| HLC 2 | _{5.62×10}6 | 99.6 | 360 | 305 |
| HLC 3 | _{6.35×10}6 | 99.3 | 320 | 306 |
| HLC 4 | _{6.18×10}6 | 99.1 | 330 | 307 |
| HLC 5 | _{3.45×10}6 | 99.6 | 580 | 308 |
| HLC 6 | _{3.72×10}6 | 99.7 | 540 | 309 |

Labeling the cells with CellPlex: The cells were labelled with molecular tags or CellPlex (according to original protocol CG000391 Cell Labeling with Cell Multiplexing Oligo RevA). Later, a specific volume of each sample was transferred into new 2 ml tubes and after labeling, the cells were washed 3 times with 2% FBS/PBS (instead of 2 times in comparison with the original protocol). After the last wash, the cells were resuspended in 600 μl of 2% FBS/PBS and counted at LUNA. Table 2 below provides data corresponding to vitality and concentration of cells post labelling.

TABLE 2
Vitality and concentration of cells post labeling

| | Concentration | Viability | μl into |
|---|---|---|---|
| Sample | [b/ml] | [%] | CellPlex staining |
| HLC 1 | _{3.35×10}6 | 99.7 | 300 |
| HLC 2 | _{3.22×10}6 | 99.7 | 312 |
| HLC 3 | _{3.44×10}6 | 99.5 | 292 |
| HLC 4 | _{3.34×10}6 | 99.7 | 300 |
| HLC 5 | _{3.15×10}6 | 99.3 | 319 |
| HLC 6 | _{3.95×10}6 | 99.4 | 255 |

The samples were pooled proportionally according to Table 2, and the final pool is passed through a 30 μm filter. Finally, the cells were counted and diluted to optimal concentration. Loading to Chromium Controller: The cells were loaded on to the Chromium Controller according to original protocol CG000390 Chromium Next GEM Single Cell3 v3.1 Cell Surface Protein Cell Multiplexing RevB. A total of 14000 cells with cell concentration of about 1600 b/μl were recovered. Subsequently, 14.4 μl cell suspension and 28.8 μl nuclease-free water (NFW) was loaded and multiplexed samples were labeled with CellPlex to result in GEL27 (Gene Expression Library) and a CSPL27 (Cell Surface Protein Library) libraries.

Library preparation: Sequencing libraries were prepared according to the original protocol CG000390 Chromium Next GEM Single Cell3 v3.1 Cell Surface Protein Cell Multiplexing RevB. In this regard, the 10× Barcoded full-length cDNA from poly-adenylated mRNA and barcoded DNA from CMP Feature Barcode were generated. The 10× Barcoded cDNA molecules were amplified using polymerase chain reaction (PCR), using compatible primers to generate sufficient mass for library construction. Moreover, the polymerization time of cDNA amplification was extended from 1 min to 1.5 min. After cDNA purification, the GEL27 sample was processed in parallel in two samples (GEL27A and GEL27B) with the modifications mentioned below. After fragmentation, double size selection was modified for samples according to Table 3 below.

TABLE 3
Double size selection using two Solid Phase
Reversible Immobilization (SPRI) steps

| | | Volume of 1. | Transfer | Volume of 2. | |
|---|---|---|---|---|---|
| Step | Sample | SPRI [μl] | volume [μl] | SPRI [μl] | |
| After | GEL27A | 20 (0.4x) | 65 | 15 | (0.7x) |
| fragmentation | GEL27B | 25 (0.5x) | 70 | 5 | (0.6x) |
| After PCR | GEL27A | 50 (0.5x) | 140 | 10 | (0.6x) |
| | GEL27B | 50 (0.5x) | 140 | 10 | (0.6x) |

After PCR amplification, both samples were purified using two Solid Phase Reversible Immobilization (SPRI) steps according to Table 3. At last, quality and quantity of libraries was determined using Fragment Analyzer and QuantiFluor dsDNA System and a quality control (QC) metrics was obtained as provided in Table 4 below.

TABLE 4
QC metrics

| | | Number of | Concentration | |
|---|---|---|---|---|
| | Concentration of | PCR | of final library | |
| Sample | CDNA [ng/μl] | cycles | [ng/μl] | Index |
| GEL27A | 9.7 | 12 | 36 | SI-GA-C8 |
| GEL27B | 9.7 | 12 | 22 | SI-GA-D8 |
| CSPL27 | 32 | 6 | 60 | SI-NN-G1 |

It will be appreciated that, in this regard, the various chemicals or kits used were Next GEM Chip G Single Cell Kit, Next GEM Single Cell 3′ Gel Beads Kit v3.1, Next GEM Single Cell 3′ GEM Kit v3.1, Dynabeads MyOne Silane, Next GEM Single Cell 3′ Library Kit v3.1, Single Index Kit T Set A, 3′ CellPlex Kit Set A, 3′ Feature Barcode Kit, and Dual Index Kit NN SetA.

### Computational Pipeline and Processes

Subsequently, the libraries are sequenced and analyzed with Cell Ranger. Additionally, after sequencing and analyzing the libraries using Cell Ranger pipeline or tool, various other pipelines or tools, namely, bamtofastq pipeline, Tizkit pipeline, Tiznit pipeline, AgeTreeShape pipeline, ReproSignal pipelen and CellTreeModel pipeline were used for generation of cell lineage trees and estimating the biological age of the biological sample based on the generated cell lineage trees.

The bamtofasto pipeline was used to convert the complex BAM files into simpler FASTQ format files containing quality scores corresponding to each nucleotide in the sequence reads.

The Tizkit pipeline was used for calling or identifying somatic SNVs from the PBMCs using the scSNV mapping tools (built by Gavin Wilson et al.; PMID 33962667). Herein, a relevant filter was set in the Tizkit pipeline, i.e. in the scSNV tool, the variant allele fraction was set to 0.75, in order to capture both frequent and rarer variants.

The Tizkit pipeline comprises 7 subsequent pipeline steps briefly summarized below as:
Counts step: for counting the number of cellular barcodes;
Map step: for mapping the reads to the genome, quantify gene expression, and writing the sorted mRNA-tag alignments;
Collapse step: for collapsing the mRNA-tags into collapsed molecules;
Pileup step: for piling-up the reads from the collapsed molecules using a list of passed barcodes;
Annotate step: for annotating the variants with information from different databases;
Count step: for quantifying SNV co-expression and collapsed molecule lengths; and
Convert step (not part of the scSNV pipeline but specifically developed): for making a matrix of the allele variants with all the information needed to apply different filters later.

Notably, the most relevant output of the Tizkit pipeline contains the alt.mtx file and the vcf files with the alignments.

The Tiznit pipeline was used for generating cell lineage trees based on the cell specific SNVs called by Tizkit. The Tiznit pipeline consists of 3 steps:
Fasta step: This step generates a fasta alignment file from all the cells and variable sites (SNVs) available. Notably, this is an optional step.
Sample Fasta step: This step generates a fasta alignment file sampling a specified number of cells or a specified list of cellular barcodes. This file will be used for the cell lineage tree generation.
Tree generation step: This step generates the cell lineage tree using the upgma method (Phylogenetics software, package, module used: the R package phangorn v2.8.1 with helper functions from the ape package v5.6.2. The substitution model used was Felsenstein's F81, and the matrix of pairwise distances was computed with the dist.dna function of the ape package. Tree inference provided rooted, ultrametric trees by default. The trees were stored in newick files). UPGMA stands for Unweighted Pair Group Method with Arithmetic mean and it is a simple agglomerative hierarchical clustering method. It needs the sample fasta file containing the multiple alignment of the different cells sampled from the individual to compute a distance matrix.

The Tiznit pipeline generates by default a rooted tree that specifies the branching topology fully and computes all the branch lengths from the number of substitutions per variable site.

The Tiznit pipeline may set relevant filters, affecting the output of the input Tizkit pipeline, as briefly mentioned below:
Min_mol: This is the minimum number of supporting molecules (sequencing reads) that were detecting the particular single nucleotide variant per cell. This is an important quality filter that reduces the number of false positive reads. The best results have been reached with this filter set to 11, requiring at least 11 molecules detecting the same somatic variant within the same cell.
Min_var: minimum number of different cells detecting the same SNV. This filter defines the minimum number of cells in the same sample/individual that a particular variant should be detected in, in order to generate a Cell Tree. The default setting used is the minimal 1, meaning the particular somatic variants used to generate the Cell Tree have been detected at least in one cell that was sampled by the Tiznit pipeline. Since the min_mol filter was set to a very high number, this filter could be kept minimal to provide a good resolution tree.
Min_cell: minimum number of cells that are sampled from all the single cell outputs per individual that are used as the tips of the phylogenetic cell lineage tree. The best results have been achieved using 700 cells or more.
Barcodes: specifying the cellular barcodes of the individual cells, whose variant information will be used for cell tree generation.

The AgeTreeShape pipeline performs two tasks, one main and one optional. It takes as input the cell lineage trees of the samples generated by the Tiznit pipeline. The main task is to compute and store multiple tree features of the cell lineage trees generated by the Tiznit pipeline in the previous step. The optional task is to calibrate linear regression plots against the age of the samples using the exhaustive list of the computed Cell Tree Topology and Branch Length features individually and then compute particular test sample Cell Tree Age estimations.

Concerning the main task below are the classified list of tree features used and instructions on how to compute them. These tree features are used to comprehensively and unbiasedly capture the properties of the cell lineage trees. The comprehensive design was to ensure to pick up the signals that could turn out to be useful when estimating the biological Cell Tree ages of the samples. For this task both brand new features have been developed and also features have been used that are already described in the literature. The literature-based features come from the general phylogenetics literature designed to understand different evolutionary processes in the tree of life. However, none of these described features have previously been used directly for cell lineage trees or for biological age estimation. This way an explorative, unbiased, but also targeted set of tree features have provided elements of the multiple regression model. The features are grouped below based on their technical properties.

### Group I. Laplacian Tree Features

There are two groups of features under this category:

I/a Features based on the non-modified Graph Laplacian (GL) matrix considering only branching order but not considering branch-length information.

I/b Features based on the Modified Graph Laplacian (MGL) matrix considering both topology and branch-length information.

I/a Features based on the non-modified graph Laplacian matrix that do not consider branch-length information.

The idea of determining the eigenvalues of a cell lineage tree and using them to understand cell tree shape and age prediction is a feature of the present disclosure. In order to determine eigenvalues one needs to choose a matrix representation of a tree, and the choice here was to generate the non-modified Graph Laplacian Matrix of a cell lineage tree (unrooted or rooted). Once such a matrix is generated and digitally stored, different ‘eigen’ properties of the matrix can be extracted from it. The following mathematical/algorithmic steps were used on an incoming cell lineage tree input stored in the newick format.
Represent the tree as a graph.
Calculate the graph Laplacian matrix, L, where L=D−A, wherein ‘D’ is the diagonal degree matrix, the matrix of node degrees, where the diagonal element i is the sum of all the nodes from node i to all the others, and ‘A’ is the adjacency matrix, where the element i of column n and column m (representing node n and node m, respectively) has weight 1 in case they are connected, and 0 otherwise. This way assigned branch lengths are ignored by the algorithm.
Calculate the eigenvalues of the graph Laplacian matrix. The Laplacian Spectrum of the tree consists of the eigenvalues and their distribution. The steps so far are necessary to generate the eigenvalues of the non-modified Laplacian matrix. It is non-modified because the actual numerical values of the calculated branch lengths are not taken into account when computing the Laplacian matrix and the eigenvalues. From this non-modified graph Laplacian Spectrum several different features can be applied to understand particular properties of the Cell Tree. Here we describe four, out of which two called ‘hic’ and ‘cso1’ are newly developed features:
‘hic’ is calculated as the number of eigenvalues of the Graph Laplacian Spectrum less than or equal with 1.0, and ‘cso1’ is calculated as the number of eigenvalues bigger than 1.0, so it is the complementary version of ‘hic’.
Algebraic Connectivity: the second smallest eigenvalue of the non modified GL matrix.
Wiener index: sum of the shortest-path distances between each pair of reachable nodes.

I/b Features based on the modified graph Laplacian matrix considering both topology and branch-length information.

The logic is the same as in the non-modified case, except the graph Laplacian matrix, L is generated by the actual numerical values of the calculated branch lengths taken into account. The following steps are performed before any of the features are computed.
Represent the tree as a graph.
Calculate the modified graph Laplacian matrix, L, where L=D-A and D is the degree matrix, the diagonal matrix and the diagonal element at i is the sum of the branch lengths from i to all the other nodes in the cell lineage tree and A is the distance matrix where the branch lengths are used as weights. This way the numerical values of assigned branch lengths are factored into the calculation and the resulting matrix.
Calculate the eigenvalues of L of the MGL.
Normalised eigenvalues: Calculate the natural log of all eigenvalues except the first one, which is 0.
A Gaussian kernel convolution turns the normalized eigenvalues into a Spectral Density Profile (SDP).

The following seven tree features can then be calculated based on the MGL:

Kurtosis: the fourth central moment divided by the square of the variance of the eigenvalues of the MGL.

Tracer: the maximum height of the Spectral Density Profile.

Skewness: the skewness statistics of the Spectral Density Profile calculated from the 3^{rd} and 2^{nd} moment of the distribution of the eigenvalues of the MGL as 3^{rd} moment/2^{nd} moment \hat{ }3/2.

mMaxEigen: the biggest eigenvalue of the MGL.

mMax_eigengap: the largest difference between two consecutive eigenvalues of the MGL.

Modified Algebraic Connectivity: the second smallest eigenvalue of the MGL.

Mode-value: The most frequent eigenvalue turned modus of the gaussian kernel.

### Group II. Clonality Focused Features

There are two groups of tree features belonging here asking about the clonality property of the trees: Fourier transform based and entropy based ones.

II/a. Fourier Transform Based Features

The features listed in this group have been developed specifically with a focus on aging characteristics through cell lineage trees. The method consists of finding the generalized Fourier transform of the cell lineage tree topology with the following steps. The Python libraries NetworkX, numpy and scipy have been used.
Find the root of the tree
Recursively find the children, assigning a 1 to children that exist and a 0 to children that could exist but do not (in a binary tree, each internal node can have 2 children)
To efficiently Fourier transform the resulting set of 1's and 0's, identify the parts of the Fourier transform matrix associated with the 1's.
The sign of each non-zero entry in the Fourier transform matrix depends on whether the cell is on the left or right side of the bifurcation associated with the entry.
Keep only the rows and columns in the transform matrix that have at least 1 non-zero value.
Sort column by labels.
Square each coefficient then sum over g, the generation of the cell
Compute actual tree features by generating different sums and average coefficients per different size of the clones involved. Coefficients are labelled by [symbol] , the generation at which the bifurcation occurs:
Avgen_10: Summing the coefficients up until [symbol] =10.
Avgen_20: Summing the coefficients up until [symbol] =20.
Avgen_40: Summing the coefficients up until [symbol] =40.
Avgen: Summing up coefficients and creating a rolling average over each spectrum in the tree.
Avgen_half_g_max: Summing up coefficients and normalising them by taking half of the depth of the particular tree.
   II/b. Entropy Based Features

The Python libraries NetworkX, numpy and scipy have been used.

### Entropy_Tips

compute the pairwise distances (branch lengths) between the tips of the cell lineage tree
make a histogram of the pairwise distances by grouping them into 50 bins, yielding a 50 element vector
compute entropy using the histogram information

### Entropy_All_Nodes

compute the pairwise distances (branch lengths) between all the nodes of the cell lineage tree
make a histogram of the pairwise distances by grouping them into 50 bins, yielding a 50 element vector
compute entropy using the histogram information

### Group III. Branch Length Focused Features

The features in this group highlight quantities based on the branch lengths in the tree.

### BranchLengthLogNorm

The sum of all the branch lengths are computed with the help of the ape R package.
The sum is divided/normalised by the natural logarithm of the number of tips of the tree.

### MeanBranchLength

The sum of all the branch lengths in the tree is computed with the help of the help of the ape R package.
The mean branch length is computed by dividing the sum of all the branch lengths with the number of all nodes in the tree.

### Group IV. Phylogenetic Features

The tree features listed here are coming from the broad phylogenetics and phylogenetic diversity literature used in traditional tree of life phylogenetic trees.

Colless index: A statistics designed to assess tree symmetry, recursively summing up the differences between left and right leaves at every stage of the tree. Only considers branching order but not branch lengths.

Sackin index: A statistics designed to assess tree symmetry, the sum of all the branches between a root and a leaf in tree, summed up for all leaves. Only considers branching order but not branch lengths.

Total cophenetic index: Sum of the branch lengths of the lowest common ancestors for all pairs of leaves in the tree. Normalised by the number of leaves in the tree.

tipRootNodes: Captures topology only information, sum of all the sums of the number of the internal nodes on the path between the leaves of the tree and the root.

tipRootPatr: Captures both topology and branch length information: sum of all the sums of the branch lenghts on the path between the leaves of the tree and the root.

tipRootSumDD: Captures topology only information, sum of all the sums of direct descendants of all nodes on the path between the leaves of the tree and the root.

### Group V. Distance Matrix Based Features

The features in this group were specifically developed with focusing on the cell lineage tree aging application.

tipDistNorm:
Distances between the tips of the tree are computed using the branch length information with the distTips function of the adephylo package.
The sum of all the distances between the tips is computed
The sum is divided/normalised by the square of the number of tips used to generate the tree
   tipDistLogNorm:
Distances between the tips of the tree are computed using the branch length information with the distTips function of the adephylo package.
The sum of all the distances between the tips is computed
The sum is divided/normalised by the natural logarithm of the number of tips used to generate the tree
   tipDistSD
Distances between the tips of the tree are computed using the branch length information with the distTips function of the adephylo package.
The standard deviance of the distances between the tips is calculated

### Group VI. Graph Features

The Python libraries NetworkX, numpy and scipy have been used. The square of a binary cell lineage tree is it's powergraph where node v and node u are adjacent if in the original tree u and v are at most two edges away from each other. Once a powergraph has been generated the Graph Laplacian Matrix can be generated similarly to Group I features above. From the Graph Laplacian the graph's algebraic connectivity can be computed which is a well-known feature of graph robustness. There are two features used here based on whether the underlying Graph Laplacian is non-modified and considers only topology information or modified and considers branch lengths as well.

AC_2: the algebraic connectivity based on the non-modified GL of the powergraph of the cell lineage tree
Generate the powergraph of the cell lineage tree
Compute the non-modified Graph Laplacian matrix of the powergraph-Calculate the eigenvalues of L of the MGL.
Normalise eigenvalues: Calculate the natural log of all eigenvalues except the first one, which is 0.
Compute Algebraic Connectivity: the second smallest eigenvalue of the non-modified GL matrix.

mAC_2: the algebraic connectivity based on the modified GL of the powergraph of the cell lineage tree
Generate the powergraph of the cell lineage tree
Compute the modified Graph Laplacian matrix of the powergraph
Calculate the eigenvalues of L of the MGL.
Normalise eigenvalues: Calculate the natural log of all eigenvalues except the first one, which is 0.
Compute Modified Algebraic Connectivity: the second smallest eigenvalue of the modified GL matrix.

Concerning the optional task, the AgeTreeShape pipeline is used for generating a simple cell lineage tree aging timer (or clock). The AgeTreeShape pipeline takes as input the cell lineage trees of the samples generated by the Tiznit pipeline. One of the input sample Trees is defined as the Test sample and the other Trees (or all Trees) are used to perform the linear regression against chronological age according with the individual Tree Features generated in the main step of AgeTreeshape before.

The ReproSignal pipeline takes as its input the numerical values of the tree features/predictors generated by AgeTreeShape from replicate trees per individual samples and computes the so-called within/between sample variation. The within/between sample variation (w/b value) is calculated by dividing the mean of variances of the tree features of the n replicates of the same individual sample showing technical variation due to the tree generating process within the same sample by the variance of the means between the replicates of different samples suggesting true biological variance between samples. Then a cutoff is set up based on the w/b value to select tree features to be used in the next step that show a good enough between-sample biological variation compared to the within-sample technical variation. This cut-off is usually 0.1 so features are selected that show w/b values of less than 0.1 meaning between sample biological variation is at least 10 times bigger than within-sample technical variation. FIG. 1 shows which 15 tree features have been selected based on the 18 samples by using 30 replicates of each sample. These 18 samples were produced according to the methodology described above.

The CellTreeModel pipeline performs the 2 crucial last procedures of Cell Tree Rings: i., it is using a penalised multiple regression algorithm to build the model out of the tree features selected by ReproSignal and ii., it estimates/predicts the actual Cell Tree Ages of the samples.

The first procedure is calculating the final aging clock and the penalised multiple regression of choice is Lasso, or L1 regularized regression. Lasso provides a regularisation constraint on minimizing the least-squares objective function to build a regularised model out of multiple features, the number of features can be larger than the number of samples, by imposing a penalty term for the number of non-zero coefficients. Lasso is implemented with LARS, which stands for Least Angle Regression. LARS is a homotopy approach, a computationally efficient way to solve lasso and produces the entire solution path as a function of the regularisation parameter. We have used the linear model. LassoLarsCV module of scikit-learn in python to build the model used for prediction in the second step of CellTreeModel. Briefly, the robust set of tree features showing true biological variation between samples were selected by the ReproSignal pipeline, and they were used together with the binary sex variable to do leave-one-out cross-validation to estimate the alpha regularisation parameter providing the minimum RMSE (root mean square) error. To capture nonlinear features, polynomial interaction terms have been used between the features. This cross-validation procedure trained the model (the aging clock). Once an aging signal has been established reproducibly with the model built using 30 replicates of all the 18 samples, the next step was to reduce the number of features used in the model by filtering out heavily correlated features. This way the model has been reduced from 16 features down to 10 features. Reproducibility of the result has been confirmed again with these smaller set of features. The next and final step before actual age prediction was to select and keep only those features that actually contributed the bulk of the non-zero coefficients during cross-validation. This way our final model contained only 3 features, BranchLengthLogNorm, Kurtosis and Sex, as these have contributed by far the biggest numerical values to the actual coefficients.

The second component of the CellTreeModel pipeline was then using these three features selected in the previous step to predict/estimate the Cell Tree Ages of the original samples. During the prediction step for training data, mean values across replicates are used as the predictor and response for each individual. This training data is then used to predict the Cell Tree biological age of all the replicates of the test individuals left out of the training data. The final Cell Tree Age of the individuals is established by averaging the predicted Cell Tree Ages across the particular replicates belonging to the same individual. Then error terms are estimated in the test set by comparing the Cell Tree Ages to the actual chronological ages. FIG. 2 shows a scatter plot of the Cell Tree Age test prediction on 18 human blood samples. The x axis is Chronological Age in years, and the y axis shows the numerical values of the predicted Cell Tree Ages in years. The red diagonal dashed line is for display purposes only. The error term indicated is the Median Absolute Error, and the numerical value of the error is 6.96 years. Table 5 below shows the Chronological Ages and the actual predicted Cell Tree Ages of the test individuals in table format.

TABLE 5
Cell Tree Age Prediction

| | Predicted Cell |
|---|---|
| Chronological Age | Tree Age |
| 21 | 36.6 |
| 24 | 36.4 |
| 25 | 35.8 |
| 29 | 27.4 |
| 32 | 35.1 |
| 37 | 36.7 |
| 37 | 42.5 |
| 41 | 30.9 |
| 42 | 53.3 |
| 47 | 47.4 |
| 51 | 45.7 |
| 53 | 55.0 |
| 54 | 65.1 |
| 62 | 65.4 |
| 65 | 56.6 |
| 75 | 53.0 |
| 78 | 77.7 |
| 82 | 53.1 |

To show the three possible relations between Cell Tree Age and Chronological Age, three actual test results are discussed below.
i., As it can be seen in Table 5 above, the Predicted Cell Tree Age of the sample coming from a 47 year old individual is 47.4 years indicating that the chronological age and the biological estimated age are synchronized, in the same range in consideration with the error term, with each other.
ii., As it can be seen in Table 5 above, the Predicted Cell Tree Age of the sample coming from a 54 year old individual is 65.1 years indicating that the chronological age and the biological estimated age are disconnected from each other and the difference between the two is higher than the computed median absolute error. Hence, the conclusion is that based on this calibration the estimated biological Cell Tree Age is more advanced and provides an indication of accelerated biological aging in the peripheral blood tissue.
iii., As it can be seen in Table 5 above, the Predicted Cell Tree Age of the sample coming from a 75 year old individual is 53 years indicating that the chronological age and the biological estimated age are disconnected from each other and the difference between the two (22 years) is at least three times higher than the computed median absolute error margin of 6.96 years. Hence, the conclusion is that based on this calibration the estimated biological Cell Tree Age is smaller/younger than the Chronological Age and this can be interpreted as an indication of decelerated biological aging in the peripheral blood tissue of this individual, which is the favourable outcome.

1-20. (canceled)

21. A method for calculating a biological age of a biological sample, the method comprising:
collecting the biological sample from a subject, wherein the biological sample is a peripheral or capillary blood comprising thousands to millions of cells, wherein viable peripheral blood mononuclear cells (PBMCs) or cells (as used hereafter) were isolated from the collected biological sample, wherein the collected biological sample is 4 ml of peripheral blood;
preparing a single-cell RNA (scRNA) sequencing library from the biological sample, wherein preparing the scRNA sequencing library comprises a polymerization process and a purification process comprising two or more purification steps;
identifying at least one genetic difference in the biological sample from the single-cell RNA (scRNA) sequencing library, wherein identifying the genetic difference employs a scRNA SNV mapping tool with a variant allele fraction threshold of 0.75, requiring at least 11 molecules detecting the same somatic variant within the same cell;
establishing filters to maximise true positive somatic mutation calls in the single-cell RNA (scRNA) sequencing library;
generating a cell lineage tree that reflects a branching order of cells of the biological sample, wherein the branching order of cells provides a data set for validating the biological age of the biological sample, and wherein the cell lineage tree is generated using a Tiznit pipeline that applies hierarchical clustering (UPGMA method);
wherein the method employs at least one algorithm selected from: a linear regression, a penalized multiple regression, the scRNA SNV mapping tool, a Laplacian transform, a Wavelet based transform, Fourier based transform, a distance matrix-based algorithm, wherein each of the linear regression, the multiple regression comprises a comprehensive list of tree features used together as multiple predictor variables and a dependent response variable.

22. The method according to claim 21, wherein the polymerization time is in a range of 1-2 minutes.

23. The method according to claim 21, wherein the at least one genetic difference is selected from somatic single nucleotide variants (SNV) from a single-cell RNA (scRNA) sequencing library.

24. (canceled)

25. (canceled)

26. The method according to claim 21, further comprising identifying a root of the cell lineage tree.

27. The method according to claim 21, wherein the data set for calculating the biological age comprises at least one of: a population history, a temporal history.

28. The method according to claim 21, wherein validating the biological age of the biological sample is based on a calibration curve obtained using the data set.

29. The method according to claim 21, wherein the scRNA sequencing library comprises at least one of: a gene expression library, a cell surface protein library, any other library, for generating a high-resolution cell lineage tree.

30. (canceled)

31. A cell lineage tree based aging timer for calculating a biological age of a biological sample using the method of claim 21, the cell lineage tree based aging timer configured to identify at least one genetic difference in the biological sample based on a data set obtained from a branching order of cells of the biological sample.

32. The cell lineage tree based aging timer according to claim 31, wherein the at least one genetic difference is selected from somatic single nucleotide variants (SNV) from a single-cell RNA (scRNA) sequencing library prepared using the method of claim 21.

33. (canceled)

34. The cell lineage tree based aging timer according to claim 23, wherein each of the linear regression and the multiple regression comprises an independent predictor variable and a dependent response variable.

35. The cell lineage tree based aging timer according to claim 33, further comprising employing the at least one algorithm to process the data set and represent the calculated biological age of the biological sample as a graphical representation.

36. The cell lineage tree based aging timer according to claim 31, wherein the data set for calculating the biological age comprises at least one of: a population history, a temporal history.

37. The cell lineage tree based aging timer according to claim 31, wherein validating the biological age of the biological sample is based on a calibration curve obtained using the data set.

38. The cell lineage tree based aging timer according to claim 32, wherein the scRNA sequencing library comprises at least one of: a gene expression library, a cell surface protein library, any other library for generating a high-resolution cell lineage tree, wherein the high-resolution cell lineage tree is used for producing the cell lineage tree based aging timer.

39. (canceled)

40. A computer program product comprising a non-transitory computer readable storage medium having computer-readable instructions stored thereon, the computer-readable instructions being executable by a computing device comprising a processor to execute the method as claimed in claim 21.
